(19) United States
(12) Patent Application Publication
(10) Pub. No.: US 2022/0083884 A1
(43) Pub. Date: Mar. 17, 2022

(54) ESTIMATING LATENT REWARD FUNCTIONS FROM EXPERIENCES

(71) Applicant: Mayo Foundation for Medical Education and Research, Rochester, MN (US)

(72) Inventors: Nicholas Chia, Rochester, MN (US); Iman J. Kalantari, Rochester, MN (US)

(21) Appl. No.: 17/424,398

(22) PCT Filed: Jan. 10, 2020
(86) PCT No.: PCT/US20/13068

§ 371 Date: Jul. 20, 2021

Related U.S. Application Data
(60) Provisional application No. 62/797,775, filed on Jan. 28, 2019.

(51) Int. Cl. G06N 7/00; G06N 3/12; G06K 9/62

(52) U.S. Cl. CPC G06N 7/005; G06K 9/6262; G06N 3/126

(57) ABSTRACT

Methods, systems, and apparatus, including computer programs encoded on a computer storage medium, for estimating latent reward functions from a set of experiences each experience specifying a respective sequence of state transitions of an environment being interacted with by an agent that is controlled using a respective latent policy. In one aspect, a method includes: generating a current Markov Decision Process (MDP); initializing a current assignment which assigns the set of experiences into a first number of partitions that are each associated with a respective latent reward function; updating the current assignment, including, for each experience: selecting a partition from a second number of candidate partitions; and assigning the experience to the selected partition; and updating the latent reward functions in accordance with a specified update rule; and updating the current MDP using latent features associated with particular latent reward functions that are determined to have highest posterior probability.

### CROSS-REFERENCE TO RELATED APPLICATIONS

This application claims the benefit of priority to U.S. Application Ser. No. 62/797,775, filed Jan. 28, 2019, the entire contents of which are incorporated by reference in their entirety into the present disclosure.

### BACKGROUND

This specification relates to inverse reinforcement learning.

In a reinforcement learning system, an agent interacts with an environment by performing actions that are selected by the reinforcement learning system in response to receiving observations that characterize the current state of the environment. The agent receives corresponding rewards as a result of performing the actions. Some reinforcement learning systems select the action to be performed by the agent in response to receiving a given observation by following one or more policies.

An inverse reinforcement learning system can estimate such rewards or policies from data characterizing respective sequences of state transitions of the environment.

### SUMMARY

In general, one innovative aspect of the subject matter described in this specification can be embodied in methods for estimating latent reward functions from a set of experiences, wherein each experience specifies a respective sequence of state transitions of an environment being interacted with by an agent that is controlled using a respective latent policy, and wherein each latent reward function specifies a corresponding reward to be received by the agent by performing a respective action at each state of the environment, where the methods include the actions of: at each of a first plurality of steps: (i) generating a current Markov Decision Process (MDP) for use in characterizing the environment; (ii) initializing a current assignment which assigns the set of experiences into a first number of partitions that are each associated with a respective latent reward function; (iii) at each of a second plurality of steps: (a) updating the current assignment, comprising, for each experience: selecting a partition from a second number of partitions by prioritizing for selection partitions which no experience is currently assigned to; and assigning the experience to the selected partition; and (b) updating, based on the updated current assignment, the latent reward functions in accordance with a specified gradient update rule; and (iv) updating the current MDP using latent features associated with particular latent reward functions that are determined to have highest posterior probability.

Other embodiments of this aspect include corresponding computer systems, apparatus, and computer programs recorded on one or more computer storage devices, each configured to perform the actions of the methods. A system of one or more computers can be configured to perform particular operations or actions by virtue of software, firmware, hardware, or any combination thereof installed on the system that in operation may cause the system to perform the actions. One or more computer programs can be configured to perform particular operations or actions by virtue of including instructions that, when executed by data processing apparatus, cause the apparatus to perform the actions.

Implementations can include one or more of the following features. In some implementations, generating the current Markov Decision Process (MDP) includes: setting the current MDP to be the same as a MDP from a preceding step in the first plurality of steps.

In some implementations, the methods further include, for a first step in the first plurality of steps: initializing a Markov Decision Process (MDP) with some measure of randomness.

In some implementations, the second number of partitions include at least one empty partition which no experience is currently assigned to.

In some implementations, selecting the partition from the second number of partitions by prioritizing for selection partitions which no experience is currently assigned to includes: determining, based at least on a number of experiences that are currently assigned to the partition, a respective probability for each partition in the second number of partitions; and sampling a partition from the second number of partitions in accordance with the determined probabilities.

In some implementations, determining the respective probability for each partition in the second number of partitions includes determining a value for a discount parameter.

In some implementations, the methods further include, after performing the first plurality of steps: generating, based on the updated MDPs, an output that defines the estimated latent reward functions.

In some implementations, the output further defines the estimated latent policies.

In some implementations, the specified gradient update rule is a Langevin gradient update rule.

In some implementations, the environment is a human body; the agent is a cancer cell; and each experience specifies an evolutionary process of the cancer cell within the human body.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other reference mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an example inverse reinforcement learning system.

FIG. 2 is a flow chart of an example process for estimating latent reward functions from a set of experiences.

FIG. 3 shows summary results of PUR-IRL run on 27 CRC patient tumors. A) Heatmap of state/action pairs with highest reward values; B) Optimal paths derived from reward function with highest posterior-probability; C) Schematic presentation of the correlation between genetic changes and stages of colon cancer progression known as the “Vogelgram”.

FIG. 4 shows posterior probability of inferred reward functions during PUR-IRL iterations.

FIGS. 5A-5C shows GridWorld results.

FIG. 6 shows an example PUR-IRL algorithm for estimating latent reward functions from a set of experiences.

Like reference numbers and designations in the various drawings indicate like elements.

### DETAILED DESCRIPTION

This specification generally describes systems, methods, devices, and other techniques for estimating latent reward functions, latent policies, or both from experience data. The experience data includes a set of real experiences, simulated experiences, or both. Each experience specifies a respective sequence of state transitions of an environment being interacted with by an agent that is controlled using a respective latent policy. Each latent reward function specifies a corresponding reward to be received by the agent by performing a respective action at each state of the environment.

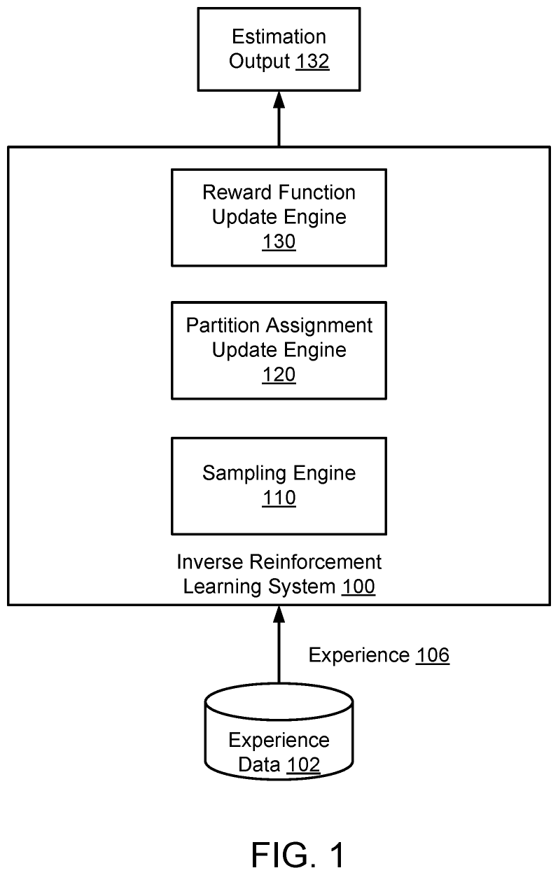

FIG. 1 shows an example inverse reinforcement learning system 100. The reinforcement learning system 100 is an example of a system implemented as computer programs on one or more computers in one or more locations, in which the systems, components, and techniques described below are implemented.

The inverse reinforcement learning system 100 is a system that receives, e.g., from a user of the system, a set of experiences 106 and processes the set of experiences 106, data derived from the set of experiences 106, or both to generate an output 132 which defines one or more estimated latent reward functions, and, optionally, one or more latent policies.

Generally, the experience data 102 characterizes agent interactions with an environment. Each experience 106, in turn, describes a sequence of state transitions of an environment being interacted with by an agent, where the state transitions are a result of the agent performing actions that cause the environment to transition states. This experience data 102 can be collected while one or more agents perform various different tasks or randomly interact with the environment. The experience data 102 can characterize, for each experience 106, both the sequence of state transitions of the environment for the experience 106 and the actions performed by the agent that caused the state transitions.

In some implementations, the environment may be a human body and the agent may be a cancer cell. In this example, the cancer cell performs actions, e.g., mutations, in order to navigate host barriers, outcompete neighboring cells, and expand spatially within the human body.

As another example, the environment may be a chemical synthesis or protein folding environment such that each state is a respective state of a protein chain or of one or more intermediates or precursor chemicals and the agent is a computer system for determining how to fold the protein chain or synthesize the chemical. In this example, the actions are possible folding actions for folding the protein chain or actions for assembling precursor chemicals/intermediates and the result to be achieved may include, e.g., folding the protein so that the protein is stable and so that it achieves a particular biological function or providing a valid synthetic route for the chemical. As another example, the agent may be a mechanical agent that performs or controls the protein folding actions or chemical synthesis steps selected by the system automatically without human interaction.

In some implementations, the environment is a real-world environment and the agent is a mechanical agent interacting with the real-world environment, e.g., a robot or an autonomous or semi-autonomous land, air, or sea vehicle navigating through the environment. In these implementations, the actions may be control inputs to control the robot, e.g., torques for the joints of the robot or higher-level control commands, or the autonomous or semi-autonomous land, air, sea vehicle, e.g., torques to the control surface or other control elements e.g. steering control elements of the vehicle, or higher-level control commands. In other words, the actions can include for example, position, velocity, or force/torque/acceleration data for one or more joints of a robot or parts of another mechanical agent. Action data may additionally or alternatively include electronic control data such as motor control data, or more generally data for controlling one or more electronic devices within the environment the control of which has an effect on the observed state of the environment. For example, in the case of an autonomous or semi-autonomous land or air or sea vehicle the actions may include actions to control navigation e.g. steering, and movement e.g., braking and/or acceleration of the vehicle.

In some implementations, the environment may be a simulated environment and the agent may be implemented as one or more computers interacting with the simulated environment. The simulated environment may be a motion simulation environment, e.g., a driving simulation or a flight simulation, and the agent may be a simulated vehicle navigating through the motion simulation. In these implementations, the actions may be control inputs to control the simulated user or simulated vehicle.

In some cases, the agent receives rewards from the environment upon performing a selected action or set of actions. The agent can receive a corresponding reward for each action that is performed by the agent, e.g., at each state of the environment. The rewards are typically task-specific. That is, agents performing different tasks within a same environment can receive different rewards from the environment.

In some cases, the agent is controlled by one or more policies. A policy specifies an action to be performed by the agent at each state of the environment. In some cases, the policy directs the agent to perform a sequence of actions in order to perform a particular task. For example, the tasks can include causing the agent, e.g., a robot, to navigate to different locations in the environment, causing the agent to locate different objects, causing the agent to pick up different objects or to move different objects to one or more specified locations, and so on.

In these cases, the policy may be an optimal policy which controls the agent to select a sequence of optimal actions which result in a highest possible total reward to be received by the agent from the environment.

However, the policies used to control the agent can be latent policies, and the rewards received by the agent can be latent rewards. In other words, the collected experience data 102 is not associated with either rewards or policies.

The system 100 can receive the set of experiences 106 in any of a variety of ways. For example, as depicted in FIG. 1, the system 100 can maintain (e.g., in a physical data storage device) experience data 102. The experience data 102 includes a set of experiences. In this example, the system 100 can also receive an input from a user specifying which data that is already maintained by the system 100 should be used as the experiences 106 for use in estimating latent reward functions. As another example, the system 100 can receive the set of experiences 106 as an upload from a user of the system, e.g., using an application programming interface (API) made available by the system 100.

In general, to make meaningful inferences about the rewards or policies from the received experiences, the system 100 uses respective Markov Decision Processes (MDPs) to model these experiences. Typically, a single MDP can be used to model many different experiences. Each MDP, in turn, defines (i) a set of possible states of an environment, (ii) a set of possible actions to be performed by an agent, and (iii) state transitions of the environment given the actions performed by the agent. Each MDP is also associated with a reward function which specifies a corresponding reward to be received by the agent by performing a respective action at each possible state of the environment.

It should be noted that, while this specification describes examples that employ Markov Decision Processes (MDPs) to characterize agent interactions with an environment, in fact, other models for sequential decision-making may also be suitable for the same purpose. The techniques described in this specification can be similarly applied to these models.

More specifically, the inverse reinforcement learning system 100 includes a sampling engine 110. The sampling engine 110 is configured to perform sampling from various data in accordance with certain sampling rules or techniques. For example, when generating respective MDPs, the system 100 can use the sampling engine 110 to select different states or actions, i.e., from a set of candidate states or actions. As another example, the system 100 can use the sampling engine 110 to generate initial latent reward functions, e.g., by selecting different rewards for different states from a plurality of possible (candidate) rewards. As another example, the system 100 can use the sampling engine 110 to generate initial assignments which assign the experiences into different partitions, e.g., by selecting, for each experience, a partition which the experience will be assigned to from a plurality of possible partitions.

After obtaining a current assignment which assigns the experiences into different partitions, the system can use a partition assignment update engine 120 to update, e.g., in an iterative manner, the current assignment. In brief, the partition assignment update engine 120 is configured to update the current assignment to determine an updated assignment for use in updating corresponding latent reward functions.

In particular, the system 100 updates the reward functions using a reward function update engine 130. The reward function update engine 130 is configured to update respective latent reward functions based on the updated current assignment and in accordance with a specified update rule. Updating assignments and latent reward functions will be described in more detail below.

Once updated, the system 100 can generate an estimation output 132 which defines these updated latent reward functions, and, optionally, latent policies which are derived from the updated latent reward functions and the experiences.

In some implementations, the system 100 can use the estimated latent reward functions and latent policies to generate simulated experiences. A simulated experience characterizes an agent interacting with an environment by selecting actions using estimated latent policies and receiving corresponding rewards specified by the estimated latent reward functions.

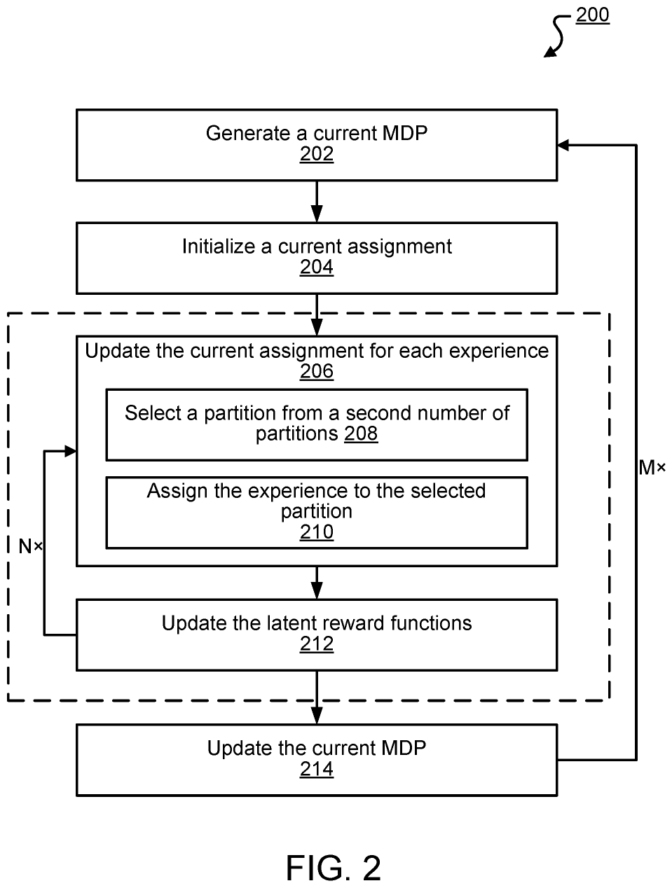

FIG. 2 is a flow chart of an example process 200 for estimating latent reward functions from a set of experiences. For convenience, the process 200 will be described as being performed by a system of one or more computers located in one or more locations. For example, a reinforcing learning system, e.g., the inverse reinforcing learning system 100 of FIG. 1, appropriately programmed in accordance with this specification, can perform the process 200.

The system receives information characterizing a set of experiences from which latent reward functions are to be estimated.

In general, the system can repeatedly perform the process 200 for the same set of experiences to generate different estimation outputs that each defines a respective latent reward function. For example, at each of a first plurality of time steps, the system can perform the process 200 to generate a respective estimation output. For example, as shown in FIG. 2, the system performs the process 200 at each of M time steps, where M is a positive integer.

The system generates a current Markov Decision Process (MDP) (202) for use in characterizing agent interactions with the environment.

At each time step after an initial time step, the system uses the MDP generated from a preceding time step (e.g., the immediately preceding time step) to update the current MDP. In other words, the system sets the current MDP to be the same as a preceding MDP from a preceding time step in the first plurality of time steps. For the very first time step in the first plurality of time steps, because there is no preceding time step, the system can instead initialize a MDP with some measure of randomness. To illustrate, the system can generate, with some measure of randomness, data defining (i) an initial set of states of an environment, (ii) an initial set of actions to be performed by an agent, and (iii) initial transitions between respective states of the environment given the respective actions to be performed at the states.

The system initializes a current assignment (204) which assigns the set of experiences into a first number of partitions. The exact values for the first number may vary, but typically, the values are smaller than the number of experiences that are received. In other words, the system assigns at least one experience into each of the first number of partitions.

The system also generates a respective initial latent reward function for each partition. The system can generate the initial latent reward functions with some measure of randomness.

Then, the system can repeatedly perform the steps 206-212 to update the latent reward functions. In other words, at each of a second plurality of time steps, the system determines a corresponding update to the latent reward functions by performing steps 206-212. For example, as shown in FIG. 2, the system can perform the steps 206-212 at each of N time steps, where Nis a positive integer which is usually different from (e.g., larger than) M.

In more detail, the system updates the current assignment (206). Updating the current assignment involves, for each experience, selecting a partition from a second number of candidate partitions (208) and assigning the experience to the selected partition (210).

Unlike the first number of partitions, the second number of candidate partitions includes empty partitions to which no experience is currently assigned. In fact, regardless of how many experiences are received by the system, the second number of candidate partitions typically includes at least one additional empty candidate partition to which no experience is currently assigned.

Step 206 can involve a Chinese Restaurant Process. For example, updating the current assignment in this manner is analogous to seating customers at an infinite number of tables in a Chinese restaurant.

In particular, for each experience, the system selects a partition from a second number of candidate partitions (208) by prioritizing for selection candidate partitions to which no experience is currently assigned. The system can do so by determining a respective probability for each candidate partition in the second number of partitions based at least on a number of experiences that are currently assigned to the candidate partition. More specifically, the system determines a respective probability for each candidate partition in the second number of candidate partitions by determining a value (e.g., between 0 and 1, either inclusive or exclusive) for a discount parameter d and concentration parameter α. The discount parameter d is used to reduce the probability for a non-empty candidate partition to be selected, whereas parameter α is used to control the concentration of mass around the mean of the Pitman-Yor process. Accordingly, the probabilities determined for non-empty candidate partitions are proportional to the number of experiences currently assigned to the candidate partition minus the value of the discount parameter d. On the other hand, the probabilities determined for empty candidate partitions are directly proportional to the value of the discount parameter d.

The system then samples a partition from the second number of candidate partitions in accordance with the determined probabilities.

The system assigns the experience to the selected partition (210).

Once the current assignment has been updated, the system then updates the latent reward functions (212) based on the updated current assignment and in accordance with a specified update rule. When the system models experiences using MDPs, the specified update rule can be any Markov Chain Monte Carlo-based update rule, for example, such as a Gibbs sampling, Metropolis-Hastings algorithm, or Langevin gradient update rule. As an advantageous implementation, updating reward functions in accordance with the Langevin gradient update rule is described in more detail in Choi, J., and Kim, K.-E. 2012. Nonparametric Bayesian inverse reinforcement learning for multiple reward functions. In Advances in Neural Information Processing Systems, 305-313, the entire contents of which are incorporated by reference into this disclosure.

The system updates the current MDP (214) using latent features associated with particular latent reward functions that are determined to have highest posterior probability. The latent features are features that characterize the respective states of the environment that are defined by the current MDP.

### Example Implementation Study

In this section, a study is described that involved an example implementation of the disclosed techniques for estimating latent reward functions and latent policies from experiences characterizing evolutionary processes of cancer cells with human bodies.

Discussion

This study explored the use of IRL as a viable approach for distilling knowledge about a complex decision-making process from ambiguous and problematic cancer data. To do so this study introduces and evaluates the PUR-IRL algorithm and its ability to use expert demonstrations of cancer evolution from patient tumor WGS data. This study demonstrates that by formalizing cancer behavior as a MDP, the state-action pairs highlighted by the inferred reward function and optimal policy can be used to reach interpretable biological conclusions. Furthermore, this study was able to show that the incremental integration of new information through iterative MDP structural updates allows for improvements in the posterior probability of the latent reward functions in an adaptive manner that is amenable to new input data. Finally, this study was able to recapitulate ground truth reward functions from simulated expert demonstrations using GridWorld, demonstrating PUR-IRL's ability to infer reward functions despite uncertainties about the source and structure of the input data.

Advantageously, these techniques can aid in the development of unreasonably effective algorithms such as PUR-IRL to further advance understanding of cancer as an evolutionary process by taking advantage of the structure and relationships that typically exist in cancer data.

Introduction

This study demonstrates the impact of considering the underlying biological processes of cancer evolution in the algorithmic design of tools for studying cancer progression. More specifically, this study demonstrates that Inverse Reinforcement Learning (IRL) is an unreasonably effective algorithm for gaining interpretable and intuitive insight about cancer progression because of its ability to take advantage of prior knowledge about the structure and source of its input data.

In support of this, this study implements the Pop-Up Restaurant for Inverse Reinforcement Learning (PUR-IRL) approach—a Bayesian nonparametric IRL model that takes advantage of relationships between events in seemingly disparate data sources by allowing for the inference of multiple reward functions from non-uniformly sampled data. In testing PUR-IRL on real-world data from colorectal cancer (CRC) patients, this study verifies its ability to infer a series of mutational events, or an evolutionary trajectory, that broadly matches those arrived at by CRC experts through the curation of a variety of multi-omics and experimental data sources. Furthermore, this study shows that PUR-IRL can accomplish this with data taken from a mere tens of patients and that this outperforms frequency-based statistical approaches that are commonly used in cancer research. Experimental results show that PUR-IRL can correctly identify the number of distinct experts, the reward function and optimal policy of each expert, and remain robust in classification under various data sampling conditions. Tested on GridWorld, PUR-IRL achieved an F1-score of 0.9328 and 0.90331 under uniform and non-uniform sampling conditions, respectively.

Methods

Data Pre-Processing

Raw Data Generation. Whole genome sequencing was performed on samples from a previous study. In brief, samples consist of normal and tumor tissue pairs from 27 patients. Sequencing was performed using the BGISEQ-500 (2×100 bp kit, ^{˜}30×) and data reads were mapped to human reference genome GRCh38 with decoy sequences. Somatic mutations and indels were determined by comparing tumor samples with normal samples using MuTect2 and subsequently filtered using FilterMutectCalls from the Genome Analysis Toolkit (GATK). Aneuploidy and somatic copy number alterations were determined using Titan and used to infer sample purity. Variant annotation was performed using SNPeff.

Extracting expert demonstrations of cancer progression from patient tumors. Tumors are comprised of multiple genetically diverse subclonal populations of cells, each harboring distinct mutations. While different subclones can appear distinct, prior knowledge tells us that they are related to one another through the process of evolution, i.e., the sequential acquisition of random mutations. Using this prior knowledge, the evolutionary relationship between these subclonal populations can be described in a series of linear and branching evolutionary expansions and modeled as a phylogenetic tree. One can assume that a cancer cell, which may exist as one of N subclones, has undergone a sequence of alterations that serve to maximize a set of rewards (i.e., growth and survival) within a competitive environment where the neighboring cancer subpopulations are competing for resources. The distinct sequence of subclones visited while traversing down from the root node down to a leaf node of a tumor's phylogenetic tree can be considered a path or expert demonstration of a cancer subclone's optimal behavior and serve as the input to the PUR-IRL algorithm.

The field of tumor phylogenetics encompasses a variety of techniques focused on the problem of subclonal reconstruction. The primary focus of such algorithms has been the deconvolution of genomic data from an observed tumor into its constituent subclones. In general, this study is not given the somatic mutations for each tumor subclone. Instead, this study has to infer these based on the variant allele fractions (VAFs) from bulk sequencing, i.e., the sum of mutations from all sub-clones within that sample. These subclonal mutations are then used to determine the phylogenetic relationships between subclones. However, these techniques have two key limitations. First, they almost never produce a unique solution. That is, for any set of genomic data extracted from a tumor, there will typically exist multiple, equally valid solutions (phylogenetic trees). Secondly, these techniques do not provide a framework for uniting disparate observations from separate tumors into a general model for understanding the drivers of carcinogenesis.

IRL methods such as PUR-IRL embrace the combinatorial explosion of paths by which each subclonal population of cancer cells may have developed by trying to unite under a single optimal policy specifying the ‘general rules’ by which cancer progresses and a reward function elucidating how the set of diverse set of state-action pairs observed across subclonal demonstrations are related.

Pop-Up Restaurant Process for Inverse Reinforcement Learning

Overview of IRL. Inverse Reinforcement Learning (IRL) infers an environment's reward function given observations of an optimally-behaving agent. Such problems can be modeled using a mathematical framework for sequential decision-making known as the Markov Decision Process (MDP). This model is defined in terms of a set of states S; a set of actions A; a stochastic transition distribution P(sₜ₊₁|aₜ, sₜ), describing the probability of outcomes following the execution of an action aₜ in state sₜ; and a reward function R(sₜ, aₜ). Given an MDP\R, inverse reinforcement learning identifies a reward function R under which π* matches the paths, where each path is a sequence of state-action pairs. In many cases, this observed behavior can be given explicitly as an optimal policy π* or as a set of sample paths generated by an agent following π*.

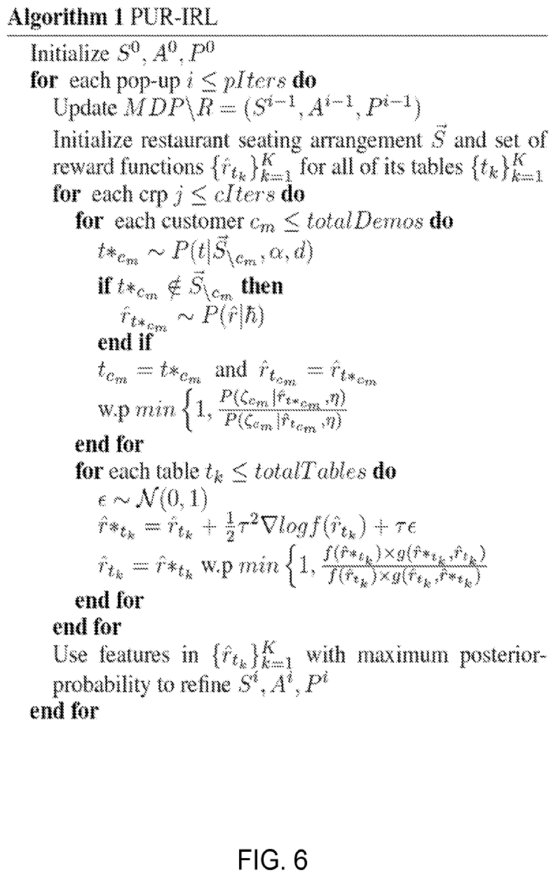

FIG. 6 shows an example PUR-IRL algorithm for estimating latent reward functions from a set of experiences.

PUR-IRL: Embracing Uncertainty during IRL. Here, this study describes a general-purpose and data-agnostic algorithm called the Pop-Up Restaurant Process for Inverse Reinforcement Learning (PUR-IRL) which can infer multiple latent reward functions from a set of expert demonstrations and use these to adapt the MDP architecture in order to integrate novel data types. The name of this algorithm alludes to the periodic updating of the MDP architecture used by the Chinese Restaurant Process (CRP). Within each periodic update, a new ‘pop-up’ CRP is used for the purpose of sampling and partitioning expert demonstrations among K MDP's, each of which with its own latent reward function rₖ. The CRP is a computationally tractable metaphor of the Polya urn scheme that uses the following analogy: consider a Chinese restaurant with an unbounded number of tables. An observation, z, corresponds to a customer entering the restaurant, and the distinct values zₖ correspond to the tables at which customers can sit. Assuming an initially empty restaurant, the CRP is expressed:

With probability proportional to cᵢ₋₁^{z}^{ₖ}−d, the i-th customer sits at the table indexed by zₖ, in which case xᵢ=zₖ, where cᵢ₋₁^{z}^{ₖ} denotes the total number of customers sitting at a table with distinct value zₖ and d is a scalar discount parameter

With probability proportional to α+Kd, the i-th customer sits at a new table, in which case xᵢ˜H, where α is a scalar concentration parameter, K is the total number of tables, and H is a random probability measure.

By using the CRP, where a Bayesian nonparametric prior represents all variables and how they relate to the data, this study can better resolve multiple probabilistic paths to cancer. In addition, the Bayesian nature of the CRP allows us to work naturally with the uncertainty of the underlying data as well as the highly skewed prevalence of events and paths in cancer patients. By applying the CRP within the IRL paradigm, this study can learn K reward functions, as K approaches infinity, from a set of data paths inferred by tumor phylogenetics.

Bayesian Nonparametric Priors in PUR-IRL. The probabilistic approach taken by PUR-IRL is similar to a previously described Bayesian nonparametric method known as the Dirichlet Process Mixture Inverse Reinforcement Learning (DPM-BIRL). Both methodologies share the notion of applying a prior on each of the reward functions \hat{r}ₜ_{ₖ} to encode preference and a likelihood to measure the compatibility of the reward function with the data, with PUR-IRL utilizing the Pitman-Yor Process (PYP) and an additional discount parameter d∈[0; 1), where d=0 reduces the model to a Dirichlet Process. Together, a and d control the formation of new reward functions.

A key property of any model based on Dirichlet or Pitman-Yor processes is that the posterior distribution provides a partition of the data into clusters, without requiring that the number of clusters be specified in advance. However, this form of Bayesian clustering imposes an implicit a priori “rich get richer” property, leading to partitions consisting of a small number of large clusters. To combat this, the use of discount parameter d is used to reduce the probability of adding a new observation to an existing cluster. The PYP prior is particularly well-suited for multi-reward function IRL applications where the set of expert-demonstrations generated by the various ground-truth reward functions may not follow a uniform distribution. The purpose of extending the IRL to use this stochastic process is to control the power law property via the discount parameter which can induce a long-tail phenomena of a distribution.

Generative Model. In PUR-IRL, the likelihood is defined as an exponential distribution that utilizes the optimal Q-function computed using reward function r and an inverse temperature parameter η that governs the exploration-exploitation tradeoff (small η>0 represents large noise, all actions are equally probable; large represents small noise and more greedy policy):

$$\begin{matrix} P ( ζ \vert \hat{r} , η ) = {\prod }_{m = 1}^{M} {\prod }_{n = 1}^{N} P ( \text{?} \vert {s}_{{c}_{m} , n} \hat{r} , η ) & ( 1 ) \\ = {\prod }_{m = 1}^{M} {\prod }_{n = 1}^{N} \frac{e \text{?}}{{\sum }_{{α}^{′}} e \text{?}} & ( 2 )\end{matrix}$$

$$\text{?} \text{indicates text missing or illegible when filed}$$

The posterior distribution can then be given by Bayes' theorem as

$${P ( \hat{r} \vert ζ , η , ℏ )}^{posterior} ∝ {P ( ζ \vert \hat{r} , η )}^{likelihood} {P ( \hat{r} \vert ℏ )}^{prior} ,$$

where h denotes hyperparameters for the prior distribution.

This study follows the CRP metaphor where the table assignment t_{c}_{ₘ}=tₖ indicates that an observed path ζ_{c}_{ₘ} belongs to the table tk. This indicates that the path is generated by the agent with reward function \hat{r}ₜ_{ₖ}. Let K approach infinity, given a set of observed agent paths represented as customers entering a restaurant ζ={ζ_{c}_{ₘ}}_{c}_{ₘ}₌₁^{M} of latent parameters θ={θ_{c}_{ₘ}}_{c}_{ₘ}₌₁^{M}, the PUR-IRL algorithm constructs a generative model in which the table t_{c}_{ₘ}=tₖ assigned to a path ζ_{c}_{ₘ} is defined by the latent parameter θ_{c}_{ₘ} drawn according to the mixture model θ_{c}_{ₘ}|G˜G, where G|α, G₀˜CRP(α; d; G₀). After the reward function \hat{r}ₜ_{ₖ} is drawn from the prior P(\hat{r}), the observed path ζ_{c}_{ₘ} is drawn from the likelihood P given by (1). The reward function can be defined as follows:

$$\begin{matrix}\hat{r} = w \cdot γ & ( 3 ) \\ R ( s , a ) = {\sum }_{f}^{F} {w}_{f} \cdot {γ}_{f} ( s , a ) , & ( 4 )\end{matrix}$$

where w: F→[0, 1] represents the weight vector sampled from the prior and γ: S×A×F→{0,1} denotes binary feature function indicating which reward features are relevant for each state-action pair. The joint posterior of the restaurant's seating arrangement \vec{S}={t_{c}_{ₘ}}ₘ₌₁^{M} and the set of reward functions {\hat{r}ₜ_{ₖ}}ₖ₌₁^{K} is defined as follows:

$$\begin{matrix}P ( \vec{S} , {\{ {\hat{r}}_{{t}_{k}} \}}_{k = 1}^{K} \vert ζ , η , α , d , ℏ ) = & ( 5 ) \\ P ( \vec{S} , \vert α , d ) {\prod }_{k = 1}^{K} P ( {\hat{r}}_{{t}_{k}} \vert {ζ}_{\vec{S} ( {t}_{k} )} , η , ℏ ) , where {ζ}_{\vec{S} ( {t}_{k} )} = \{ {ζ}_{{t}_{{c}_{m}}} \vert {t}_{{c}_{m}} = {t}_{k} for {t}_{{c}_{m}} = {t}_{{c}_{1} ,} … , {t}_{{c}_{N}} \} . & ( 6 )\end{matrix}$$

Inference Procedure. To infer the latent reward functions from a set of paths, this study approximates the full posterior joint distribution over the set of random variables via Bayesian inference with Metropolis-Hastings MCMC (MH-MCMC) sampling. MH-MCMC makes use of the full joint density function and (independent) proposal distributions for each variable of interest to simulate samples from a probability distribution. Given K unique table index values {t₁, . . . , tₖ} in the restaurant, this study can define the posterior distribution for table t_{c}_{ₘ} as:

$$\begin{matrix} P ( {t}_{{c}_{m}} \vert {\vec{S}}_{\backslash {c}_{m}} , {\{ {\hat{r}}_{{t}_{k}} \}}_{k = 1}^{K} ζ , η , α , d ) \times {P ( {ζ}_{{c}_{m}} \vert {\hat{r}}_{{c}_{m}} , η )}^{likelihood} {P ( {t}_{{c}_{m}} \vert {\vec{S}}_{\backslash {c}_{m}} , α , d )}^{prior} & ( 7 ) \\ P ( {t}_{{c}_{m}} \vert {\vec{S}}_{\backslash {c}_{m}} , α , d ) ∝ \{ \begin{matrix}\frac{count \text{/} \text{?}}{M + β} & if {t}_{{c}_{m}} = {t}_{{c}_{j}} \\ \frac{α + {K}_{d}}{M + α} & if {t}_{{c}_{m}} \neq {t}_{{c}_{j}}\end{matrix} , \\ \text{?} \text{indicates text missing or illegible when filed} & ( 8 )\end{matrix}$$

where count is the number of paths, excluding the current path, assigned to table t_{c}_{ⱼ}. Furthermore, if the sampled table t_{c}_{ₘ} for path ζ_{c}_{ₘ} assigned to a new table, a new reward function \hat{r}ₜ_{ₖ} can be drawn from the distribution:

$$P ( {\hat{r}}_{{t}_{k}} \vert \vec{S} , ( {\hat{r}}_{\backslash {t}_{k}} , ζ , η , ℏ ) ∝ {P ( {ζ}_{\vec{S} ( {t}_{k} )} \vert {\hat{r}}_{{t}_{k}} , η )}^{likelihood} {P ( {\hat{r}}_{{t}_{k}} \vert ℏ )}^{prior} \qquad ( 9 )$$

Following random initialization of restaurant seating arrangement and its corresponding reward functions, the PUR-IRL algorithm begins an iterative procedure in which it performs two update operations. In the first update operation, the seating arrangement \vec{S} is updated by sampling a new table index t*_{c}_{ₘ} for each customer cm according to Equation (7). If this new table index does not exist in the current seating arrangement \vec{S}_{c}_{ₘ}, a new reward function is drawn from the reward prior. In the second update operation, each reward function \hat{r}ₜ_{ₖ} is updated by using a Langevin gradient update rule. Following the CRP, the set of features associated with reward functions with the highest posterior probability are used for updating the S, A, P in the next pop-up restaurant iteration. Using the inferred optimal policy and reward function weights to prioritize which states and actions need to be updated, additional data sources (i.e. external functional, clinical databases, etc.) can be incrementally integrated into the MDP architecture in a tractable manner.

Real-World Use Case

The Colorectal Cancer Reward Function

This study has designed an IRL experiment that involves the reconstruction of the evolutionary trajectories of CRC directly from tumor WGS data.

Embracing Uncertainty in the MDP Structure of Cancer. Defining states and actions for IRL can be treated similarly to problems of feature representation, feature selection and feature engineering in unsupervised and supervised learning. For cancer data, this study utilizes the Generalized Latent Feature Model (GLFM). Here, a state is encoded by a binary sparse code that indicates the presence/absence of latent features, inferred via GLFM, on the nucleotide, gene, and functional pathway level. An action then represents a stochastic event such as a somatic mutation in a specific gene. In addition to generating binary codes which provide more interpretable latent profiles of states and actions in the biological domain, the GLFM's use of a stochastic prior over infinite latent feature models allows model complexity to be adjusted on the basis of observations that will increase in volume and dimensionality as new data sources are incorporated in the PUR-IRL MDP.

This study's initial MDP structure consists of 1084 actions and 144 states. An action corresponds to an event occurring at one of 1084 known driver genes of CRC aggregated from two public datasets. For example, action a₀^{AATK} corresponds to a mutation event occurring within any region of the AATK gene. The state space consists of 144 possible states composed of 12 latent features that were inferred via the GLFM algorithm. A state is an abstract representation that encodes features that are present internally or externally to a cancer cell (agent). The GLFM algorithm was used to infer these latent features from the list of alterations attributed to each inferred subclone. In this experiment, each state is represented by a 12-dimensional binary vector indicating the presence/absence of the 12 latent features inferred via the GLFM algorithm. Each latent feature reflects a unique frequency distribution of alterations to genes in 14 signaling pathways associated with CRC (Notch, Hedgehog, WNT, Chromatin Modification, Transcription, DNA damage, TGF, MAPK, STAT-JAK, PI3KAKT, RAS, Cell-cycle, Apoptosis, Mismatch Repair). To infer a set of latent features, each subclone must be converted into a 14-dimensional vector indicating the count of alterations attributed to each signaling pathway. This set of 14-dimensional vectors serves as input to the GLFM algorithm.

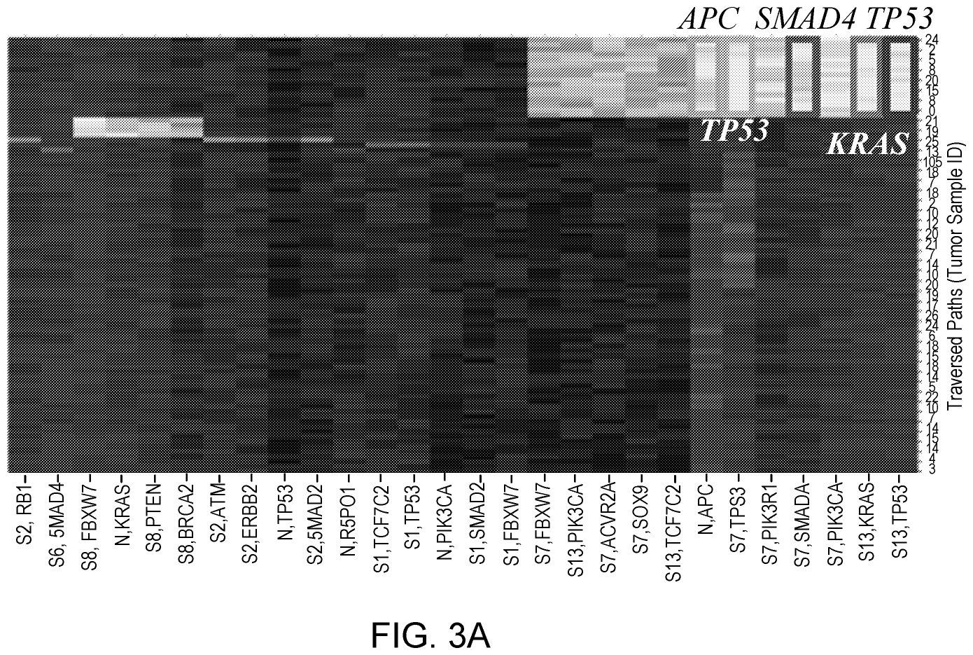

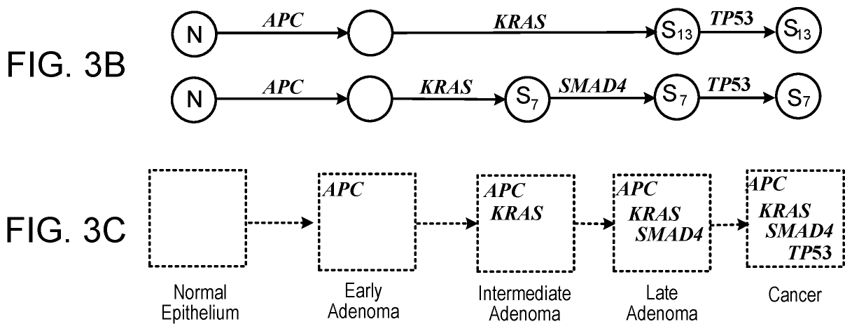

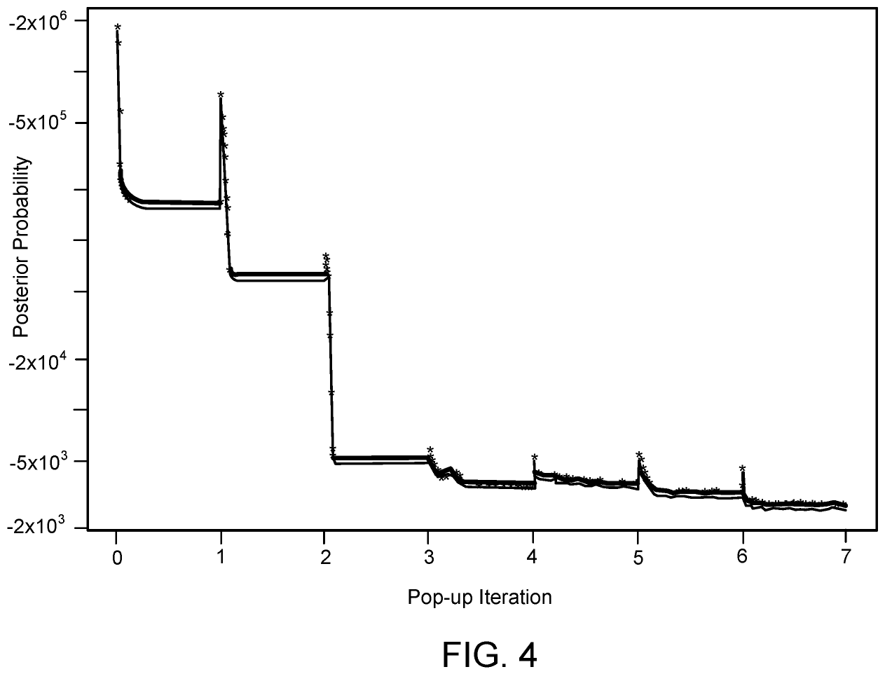

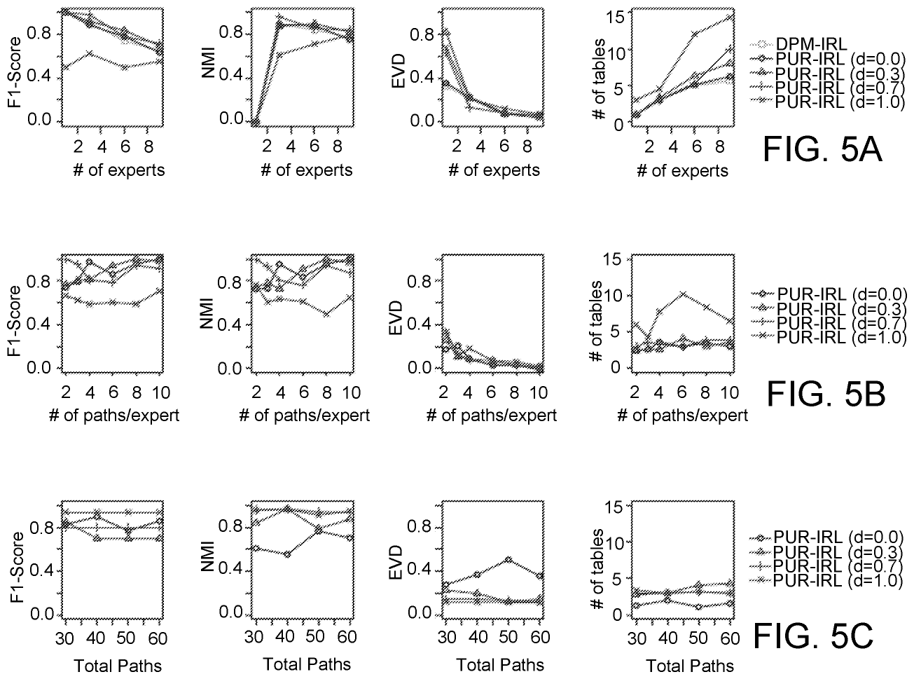

Embracing Uncertainty in Tumor Subclone Expert-Demonstrations. WGS data was used to infer the subclonal composition of tumor using a slightly modified PhyloWGS algorithm for efficiently identifying multiple possible unique phylogenetic trees. In a preliminary run of this experiment, 215,000 traversed paths derived from phylogenetic trees generated from a subset of (N=27) tumor samples were provided as expert demonstrations to the PUR-IRL algorithm; with each path describing an ordered list of subclones within a given tumor sample and represented by a corresponding sequence of state-action pairs. The PUR-IRL model was run with 6 ‘pop-up’ updates in between every 100 CRP iterations. FIG. 3 summarizes the inferred reward function with highest posterior probability from this preliminary run. FIG. 3A shows a subset of the inferred reward function across the 27 tumor dataset. The optimal policy generated over this reward function consists of the state-action pairs N-APC, S13-KRAS, S7-SMAD4, highlighted in grey, pink, and red, respectively. The actions in these pairs correspond to genetic changes that are known to characterize CRC progression as summarized in FIG. 3C. This study compares this to the most likely paths drawn in FIG. 3B that were obtained by simulating a MDP with the new reward function. Despite uncertainties in how the empirical cancer data was generated, this study was able to recapitulate an optimal path, or evolutionary trajectory, with biologically relevant conclusions which match the literature derived model of CRC progression. This demonstrates the PUR-IRL model's ability to identify singular genetic changes that are often not the most frequent, but are nonetheless critical for CRC progression. In FIG. 4, this study analyzes the posterior probability of the inferred reward functions following each of the 6 ‘pop-up’ updates and 100 intermediate CRP iterations. The results of this analysis demonstrate that incremental integration of new information by PUR-IRL provides a tractable methodology for improving the reward functions without the use of a large initial state or action-space while still allowing for the exploration of new features.

PUR-IRL Performance

In order to demonstrate the PUR-IRL algorithm's utility and accuracy, this study ran PUR-IRL on data generated by multiple experts and sampled under uniform and non-uniform sampling conditions. Specifically, this study performed three sets of experiments (130 total) that evaluated the performance of PUR-IRL on the GridWorld problem. In all experiments, this study fixed the concentration hyperparameter α=1 while evaluating different discount values {0.0; 0.3; 0.7; 1.0}, where PUR-IRL with d=0 reduces to the DPM-IRL. The GridWorld is a simple deterministic world that is often used to illustrate the basic concepts of Q-learning. It allows this study to evaluate the proposed approach under different scenarios in which the inference of latent reward functions can be validated using simulated ground-truths. In the first set of experiments, the GridWorld conditions from—3 expert demonstrations generated per expert—were amended to explicitly model 4 scenarios in which the number of experts is greater than or equal to one. In each scenario, this study randomly samples the weights for the G ground-truth reward functions (experts), from a Gaussian prior and evaluates IRL performance under uniform sampling conditions (i.e. each expert generates the same number of paths). This experiment was repeated 10 times for each scenario. The results of this experiment demonstrate that PUR-IRL and other IRL methods that use BNP priors (DPM-IRL) can recapitulate the ground-truth reward function(s) from data that follows the single expert assumption in addition to scenarios where the true number of data-generating experts is unknown. The study verified that performance of PUR-IRL (d=0) model, which reduces the underlying PYP to a DP. In the second experiment set, this study randomly sampled the weights for 3 ground-truth reward functions, under 5 uniform sampling conditions of increasing dataset size. It can be seen that with an increase in the number of paths, PUR-IRL model performance appears to improve in terms of the number of tables (inferred reward functions), normalized mutual information, F1-scores and the expected value difference (EVD) between the ground-truth reward functions and the learned reward functions. The outlier model (PUR-IRL with d=1.0) failed to improve in performance or accurately recapitulate the true number of reward functions (tables) due its heavy bias for fat-tail distributions. In the final experiment set, this study sought to evaluate IRL performance under 4 non-uniform sampling conditions which closely resemble those found in real-world data (i.e., the total set of paths is distributed across 3 experts according to a power-law distribution). This study sees that the addition of the discount hyperparameter within the PUR-IRL model allows users to control how well the final model fits with input dataset and thus allowing them to exceed performance when d=0. Although the GridWorld MDP does not encapsulate many of the complexities that this study addresses with PUR-IRL framework (i.e. ability to infer the number and identity of biologically relevant states and actions from high-dimensional data), it nevertheless demonstrates that PUR-IRL can accurately infer optimal policies and latent reward functions given a set of expert demonstrations under various data scenarios likely to be found in real-world applications.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” refers to data processing hardware and encompasses all kinds of apparatus, devices, and machines for processing data, including by way of example a programmable processor, a computer, or multiple processors or computers. The apparatus can also be, or further include, off-the-shelf or custom-made parallel processing subsystems, e.g., a GPU or another kind of special-purpose processing subsystem. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

As used in this specification, an “engine,” or “software engine,” refers to a software implemented input/output system that provides an output that is different from the input. An engine can be an encoded block of functionality, such as a library, a platform, a software development kit (“SDK”), or an object. Each engine can be implemented on any appropriate type of computing device, e.g., servers, mobile phones, tablet computers, notebook computers, music players, e-book readers, laptop or desktop computers, PDAs, smart phones, or other stationary or portable devices, that includes one or more processors and computer readable media. Additionally, two or more of the engines may be implemented on the same computing device, or on different computing devices.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and pointing device, e.g, a mouse, trackball, or a presence sensitive display or other surface by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A method of estimating latent reward functions from a set of experiences, wherein each experience specifies a respective sequence of state transitions of an environment being interacted with by an agent that is controlled using a respective latent policy, and wherein each latent reward function specifies a corresponding reward to be received by the agent by performing a respective action at each state of the environment, the method comprising:
at each of a first plurality of steps:
(i) generating a current Markov Decision Process (MDP) for use in characterizing agent interactions with the environment;
(ii) initializing a current assignment which assigns the set of experiences into a first number of partitions that are each associated with a respective latent reward function;
(iii) at each of a second plurality of steps:
(a) updating the current assignment, comprising, for each experience:
selecting a partition from a second number of candidate partitions by prioritizing for selection candidate partitions to which no experience is currently assigned; and
assigning the experience to the selected partition; and
(b) updating, based on the updated current assignment, the latent reward functions in accordance with a specified update rule; and
(iv) updating the current MDP using latent features associated with particular latent reward functions that are determined to have highest posterior probability.

2. The method of claim 1, wherein generating the current Markov Decision Process (MDP) comprises:
setting the current MDP to be the same as a MDP from a preceding step in the first plurality of steps.

3. The method of claim 1, further comprising, for a first step in the first plurality of steps:
initializing a Markov Decision Process (MDP) with some measure of randomness.

4. The method of claim 1, wherein:
the second number of candidate partitions comprise at least one empty partition to which no experience is currently assigned.

5. The method of claim 1, wherein selecting the partition from the second number of candidate partitions by prioritizing for selection candidate partitions to which no experience is currently assigned comprises:
determining, based at least on a number of experiences that are currently assigned to the partition, a respective probability for each candidate partition in the second number of candidate partitions; and
sampling a partition from the second number of candidate partitions in accordance with the determined probabilities.

6. The method of claim 1, wherein:
determining the respective probability for each candidate partition in the second number of partitions comprises determining a value for a discount parameter and concentration parameter.

7. The method of claim 1, further comprising, after performing the first plurality of steps:
generating, based on the updated MDPs, an output that defines the estimated latent reward functions.

8. The method of claim 1, wherein the output further defines the estimated latent policies.

9. The method of claim 1, wherein the specified update rule is a Langevin gradient update rule.

10. The method of claim 1, wherein:
the environment is a human body;
the agent is a cancer cell; and
each experience specifies an evolutionary process of the cancer cell within the human body.

11. A system comprising:
a data processing apparatus; and
one or more computer-readable media having instructions stored thereon that, when executed by the data processing apparatus, cause the data processing apparatus to perform operations for estimating latent reward functions from a set of experiences, wherein each experience specifies a respective sequence of state transitions of an environment being interacted with by an agent that is controlled using a respective latent policy, and wherein each latent reward function specifies a corresponding reward to be received by the agent by performing a respective action at each state of the environment, the operations comprising:
at each of a first plurality of steps:
(i) generating a current Markov Decision Process (MDP) for use in characterizing agent interactions with the environment;
(ii) initializing a current assignment which assigns the set of experiences into a first number of partitions that are each associated with a respective latent reward function;
(iii) at each of a second plurality of steps:
(a) updating the current assignment, comprising, for each experience:
selecting a partition from a second number of candidate partitions by prioritizing for selection candidate partitions to which no experience is currently assigned; and
assigning the experience to the selected partition; and
(b) updating, based on the updated current assignment, the latent reward functions in accordance with a specified update rule; and
(iv) updating the current MDP using latent features associated with particular latent reward functions that are determined to have highest posterior probability.

12. The system of claim 11, wherein generating the current Markov Decision Process (MDP) comprises:
setting the current MDP to be the same as a MDP from a preceding step in the first plurality of steps.

13. The system of claim 12, wherein the operations further comprise, for a first step in the first plurality of steps:
initializing a Markov Decision Process (MDP) with some measure of randomness.

14. The system of claim 11, wherein:
the second number of candidate partitions comprise at least one empty partition to which no experience is currently assigned.

15. The system of claim 11, wherein selecting the partition from the second number of candidate partitions by prioritizing for selection candidate partitions to which no experience is currently assigned comprises:
determining, based at least on a number of experiences that are currently assigned to the partition, a respective probability for each candidate partition in the second number of candidate partitions; and
sampling a partition from the second number of candidate partitions in accordance with the determined probabilities.

16. The system of claim 15, wherein:
determining the respective probability for each candidate partition in the second number of partitions comprises determining a value for a discount parameter.

17. The system of claim 11, wherein the operations further comprise, after performing the first plurality of steps:
generating, based on the updated MDPs, an output that defines the estimated latent reward functions.

18. The system of claim 11, wherein the output further defines the estimated latent policies.

19. The system of claim 11, wherein the specified update rule is a Langevin gradient update rule.

20. The system of claim 11, wherein:
the environment is a human body;
the agent is a cancer cell; and
each experience specifies an evolutionary process of the cancer cell within the human body.

21. One or more non-transitory computer-readable media having instructions stored thereon that, when executed by data processing apparatus, cause the data processing apparatus to perform operations for estimating latent reward functions from a set of experiences, wherein each experience specifies a respective sequence of state transitions of an environment being interacted with by an agent that is controlled using a respective latent policy, and wherein each latent reward function specifies a corresponding reward to be received by the agent by performing a respective action at each state of the environment, the operations comprising:
at each of a first plurality of steps:
(i) generating a current Markov Decision Process (MDP) for use in characterizing agent interactions with the environment;
(ii) initializing a current assignment which assigns the set of experiences into a first number of partitions that are each associated with a respective latent reward function;
(iii) at each of a second plurality of steps:
(a) updating the current assignment, comprising, for each experience:
selecting a partition from a second number of candidate partitions by prioritizing for selection candidate partitions to which no experience is currently assigned; and
assigning the experience to the selected partition; and
(b) updating, based on the updated current assignment, the latent reward functions in accordance with a specified update rule; and
(iv) updating the current MDP using latent features associated with particular latent reward functions that are determined to have highest posterior probability.
